SEQUENCE LISTING (19 sequences)

SEQ ID NO: 1 - DNA, 7193 bp, S. Clavuligerus
ccatggcgggcggcggctgccccggagcctcggccggaccggtgaccaggaccaccccggtgggatagtggcccgccacccggcgcagcagactcccggacacggacccgtgggtgtgcgcggaaaggcccggaggccgggtcacagccacgggtaacgcgcggtgtccttgcccgcgtaatcggggtccagatagacgaaggcccggtggacgaggaagtcccgcacctcgtagaccgtgcaccagcgcccggcggcccactcggggtcacccgcccgccacggcccgtcccggtgctcaccgtgggtggtgccctccgcggcgaggagttcggtcccggtcagaatccagttgacggaccacagatggtgggtgatcgagcggatggtgcccccgaggtcgtcgaagagccgggcgatctcggacttgccccgggccagaccccacttggggaagaagaagaccgcgtcctcggcgaagtagtcgatcgcgggggtgccgtcgctgccgacgccgccgttgtcgaacgccttgaagtacgcggtgatgaccgccttgcgctgctcgtccgtcataccggccgatgccacggacatgaaacgacctccagagattccgggtggctgtgctggggctgcggaaggggtgtcccccgcgaaggacggcggacgccgcggacgccgcggccgtctccccggcggacgggtcccagcgtcctggagagggcttggcggcggcttgacgccgtgctgtcccgcggcttgcggaacgcgaagtaccggccagcgtacgggcgttgcaccggacgtgtacgccggtcgggacccctcgtacccccggagccggccgaccccggcggctccgggggtacggacgcgccggaccggcccgagcgagccggacgggtcggacggtgcgcgtggttccggtgtgtcggacagctcggacggaccggacggtgcgcgtggttccggtgtgtcggacagctcggacgggtcggacggtgcgcgtggttccggcacgccggacgggtcagttgccgatcatggcgagcaatgccggggtgtaccgctccccggacaccgggtgggagatcgcggccgtcacctccgcgagggaccggtcgtccagccggatcgaggcggcggcgagattgtccgcgagatgggccgggttcgcggtgcccgggatcgggacgacgtcctcgccccggtggtgcagccaggcgagcgcgagctgtgccagggtcagccccagaccgtccgcgaccgggcgcagccggtgcagcaacgagcggttgcgcgcgagggccggagcgctgaaccggggctggccccggcggaagtcctcgtcccccagatcgtcggtggtgcggatggtgccggtgagaaaaccccgtcccagaggggcgtaagcgacgatcccgatccccagctcccggcagacgggcaccacctcgtcctcgatcccgcgcgaccacaggctccactcgctctgcaccgccgtcaccgggtgcaccgcgtccgcccggcgcagcgtggccgcggagggctcggagagaccgagcctgcggaccttgccctcgcgcaccagctcggccaccgcacccacggtctcctcgatcggcaccgccgggtccgtccagtgctggtagtacaggtcgatgcggtcggtgccgagacgacgcagggaccgttcgcaggccgcgcggacgtaggacggctcgccgcacaagccctgggaggcgccgtcggacgagcgcaccatgccgaacttggtggcgatcagcacctcgtcccggcggcccgcgaccgcccgtccgagcagctcctcaccggcgccgagcccctggacgtcggcggtgtccagcagggtgaccccggcgtcgacggcggcgcggatggtggccgtcgcccgggcgcggtccgggcgtccgtagaagtcggtggtcggcaggcagccgagcccctgggcactgaccggaaggtcccgcagggcgcggaccggcggacgcggaaccgcggcggacacggaaccggccggggactcgggcggagagcgggacatacggaacctccacaggcggagccgggaacgggacgagggcgaggacgggacggaacgaaggagaggacgggacggacagcacggacgggacggacggaacggagtcgggaaccggggggggtgaccggaaccgggccgtccttggccctcccccgtcctccccgccatccgccgttctcccccgttccctctcccgtcctccagccaacaccgccgccctttccaagcgcttgacacggcaccgacagccgccgccgggcgcccgatggggacccgtgcccgccggtgagcggcggtgagcgccggtacgggaccccacgcgccgccgcccgggcgcccgccagggcccgcgcggccaccccggcccgccccggccggagcggcgatccgggccgctcgctgcaagaggaacatccacagccgcacaaggagcgctccgcacagtgggcaccacgtccgccccgtcccccacaccgtggccggtccccaccggacagcacagcaccgcacagcaccacatcgcacggcacagcacagcaccaccggcacgaggaaccaaggaaaggaaccacaccaccatgacctcagtggactgcaccgcgtacggccccgagctgcgcgcgctcgccgcccggctgccccggaccccccgggccgacctgtacgccttcctggacgccgcgcacacagccgccgcctcgctccccggcgccctcgccaccgcgctggacaccttcaacgccgagggcagcgaggacggccatctgctgctgcgcggcctcccggtggaggccgacgccgacctccccaccaccccgagcagcaccccggcgcccgaggaccgctccctgctgaccatggaggccatgctcggactggtgggccgccggctcggtctgcacacggggtaccgggagctgcgctcgggcacggtctaccacgacgtgtacccgtcgcccggcgcgcaccacctgtcctcggagacctccgagacgctgctggagttccacacggagatggcctaccaccggctccagccgaactacgtcatgctggcctgctcccgggccgaccacgagcgcacggcggccacactcgtcgcctcggtccgcaaggcgctgcccctgctggacgagaggacccgggcccggctcctcgaccggaggatgccctgctgcgtggatgtggccttccgcggcggggtggacgacccgggcgccatcgcccaggtcaaaccgctctacggggacgcggacgatcccttcctcgggtacgaccgcgagctgctggcgccggaggaccccgcggacaaggaggccgtcgccgccctgtccaaggcgctcgacgaggtcacggaggcggtgtatctggagcccggcgatctgctgatcgtcgacaacttccgcaccacgcacgcgcggacgccgttctcgccccgctgggacgggaaggaccgctggctgcaccgcgtctacatccgcaccgaccgcaatggacagctctccggcggcgagcgcgcgggcgacgtcgtcgccttcacaccgcgcggctgagctcccgggtccgacaccgcgcggctgaacccacggtccggggcccacggtccggcaccgcgcggctgagcccccgggtccggcagcgggcggctgaacccccgccccgggccaccgcccgaccgcccccgcgcaccggacgcgcccgcctgtacggcggtcccgcccgggcccgtacacctgaagcgcccggcggaccgccgccccgccgggggacggacagagccgggtgcgggaggacgtcctcccgcacccggctcccaccgttccgcaccgaccgcacccgaccgtgccgcaggcgccaccggcaccgcaccgcccgcgccggcagccaccacaggcgccacgccgcccgcacggtgcccgcgctgctcagcccccgtccaccgggctgtccagcagccgccgcagcgcgcccccgatgaactcccggtcggcggccgaccccccggaccccgcgagatgcccccacactcccgggatcacctccagcgaggcatacggcagcagatcggccacccgcttctcgtcctcgacggcgaaacacacgtccagggcgcccggcagcaccacggcccgcgccgtgacggaggccagcgccgcctcgacgctccccccggccccgggtgtcgcccccacatccgtgttctcccaggtgcgcaccatggtgagcagatccgcggcgccgggcccggagaggaagacctgctcccagaagccggtgaggtactcctcgcgggtggcgaaacccagctcccggtgggcacggcgggcccagaaggaacgcgaggtcccccacccggcgaacacccggcccgccgccttccgcccccgctccccggcgtcggcgctgagcgccgcggccagaccggacagcaggaccaggctgtgcgggctgctcaccggcgccccgcagatcggggcgatccggcgcaccatccccggatgcgacacggcccactggtaggcgtgggccgcgcccatcgaccagcccgtgaccagggccagttcccgtacccccagctcctcggtgagcagccggtgctgcgccgcgacattgtcctgcggagtgatcagcggaaagcgggaccccgacgggtggttgccgggcgagctggagaccccgttgccgaagagtccggcggtgacgacgcagtaccgccgggtgtccagcggcagccccgcaccgatcagccagtcgtacccggtgtggtcccggccgaagaacgacggacagagcaccacgttcgtcccgtcggcgttcggcgtgccgtacatggcgtaaccgatccgggcgtcccgcaggacctccccgtccagcaacggcagttcgtcgatctcgaatatgcggcattccaccgctgacctccttgttcgatccccccggacaacaggtcggtcgtggccggagactcagagccagttgggggcgatctcggtggcccacagctccaggctgcgcagctggacatcgtgcgggatcagcccggagtactggcactggagcagatactccggatcgtgccgctccaccagcttctcgatcatgcggttgatgtcgtccggggtgccgacccactccagcccccggtcgaccagggtcttgtagtccgagccgatcggacccgtctcgccggtcgcgcgcagcgcctcggtgaagcccatggggccgaaccagttctcgaagatgaagccgccgccgcgggacgcccagtggtgggcctcgccggagtcccgggagaccaggacgtccttcatcaccccgacccgctcgccccgccgcagggtgccgtggcccgccgcctcggcctcctcccggtagatgtccatcagccgggcgacgatctggtcgtcggtgttcatcaggatcggcaccacgccctcccgggcacagaaccggaacgtgtcctcactgaagctgaacggctggaagacgggcgggtgggggcgctggtagggcttgggcgcgatgcccacctcgcggatgacgccgttctcgtcgaggccccggccgtagcggcgcaccgcctcgtaggggaactccaggtccggcaccgggatcgtccactgctccccggagtgggtgaacgtctcggtcgtccacgccttcttgatgatctcccagtgctcctcgaagagggcacgattgcgccggtcccgctccccggcgtcggacagggtgccgccgaccccgtacacctgccccatgatgtcggcccagcgcttctggaacccgcgcgcgatcccgacgaaggcgcggccccgggtcatgtggtcgagcatcgccagatcctcggccagccgcagcggattgtgcagcggcaggacgttggccatctggccgacccggatgtgccgggtctgcatgccgaggtagagccccagcatgatcgggttgttggagacctcgaaaccctcggtgtggaagtggtgctcggtgaaggacagtccccagtagccgagttcgtcggccgcctgcgcctgccgggtgagctgccggagcatgttctggtagttctgcggattgacccccgccataccccgctggacctgcgcatgactgccgaccgttggcagatagaagagaatggacttcaccctggctcctccggttcgcggcgccctccattgacgtgcgccgaaagcggctcgaccgtcccactccgcccttgagttccgtctgacgccgcgccagtcggcgggccgtccgccggggtgcccgccggggtccgcacccgccggacggcacggcgcgcaccgcgcgcgcggcgcttcggggcaccgggctcgacggggtgctcagcgggacgtccaacggaaggcaagcccccgtacccagcctggtcaaggcgctcatcgccattccctgaggaggtcccgccttgaccacagcaatctccgcgctcccgaccgtgcccggctccggactcgaagcactggaccgtgccaccctcatccaccccaccctctccggaaacaccgcggaacggatcgtgctgacctcggggtccggcagccgggtccgcgacaccgacggccgggagtacctggacgcgagcgccgtcctcggggtgacccaggtgggccacggccgggccgagctggcccgggtcgcggccgagcagatggcccggctggagtacttccacacctgggggacgatcagcaacgaccgggcggtggagctggcggcacggctggtggggctgagcccggagccgctgacccgcgtctacttcaccagcggcggggccgagggcaacgagatcgccctgcggatggcccggctctaccaccaccggcgcggggagtccgcccgtacctggatactctcccgccggtcggcctaccacggcgtcggatacggcagcggcggcgtcaccggcttccccgcctaccaccagggcttcggcccctccctcccggacgtcgacttcctgaccccgccgcagccctaccgccgggagctgttcgccggttccgacgtcaccgacttctgcctcgccgaactgcgcgagaccatcgaccggatcggcccggagcggatcgcggcgatgatcggcgagccgatc

SEQ ID NO: 2 - DNA, 145 bp, S. Clavuligerus
gtgacccggcctccgggcctttccgcgcacacccacgggtccgtgtccgggagtctgctgcgccgggtggcgggccactatcccaccggggtggtcctggtcaccggtccggccgaggctccggggcagccgccgcccgccatgg

SEQ ID NO: 3 - DNA, 453 bp, S. Clavuligerus
atgtccgtggcatcggccggtatgacggacgagcagcgcaaggcggtcatcaccgcgtacttcaaggcgttcgacaacggcggcgtcggcagcgacggcacccccgcgatcgactacttcgccgaggacgcggtcttcttcttccccaagtggggtctggcccggggcaagtccgagatcgcccggctcttcgacgacctcgggggcaccatccgctcgatcacccaccatctgtggtccgtcaactggattctgaccgggaccgaactcctcgccgcggagggcaccacccacggtgagcaccgggacgggccgtggcgggcgggtgaccccgagtgggccgccgggcgctggtgcacggtctacgaggtgcgggacttcctcgtccaccgggccttcgtctatctggaccccgattacgcgggcaaggacaccgcgcgttacccgtggctg

SEQ ID NO: 4 - DNA, 1032 bp, S. Clavuligerus
atgtcccgctctccgcccgagtccccggccggttccgtgtccgccgcggttccgcgtccgccggtccgcgccctgcgggaccttccggtcagtgcccaggggctcggctgcctgccgaccaccgacttctacggacgcccggaccgcgcccgggcgacggccaccatccgcgccgccgtcgacgccggggtcaccctgctggacaccgccgacgtccaggggctcggcgccggtgaggagctgctcggacgggcggtcgcgggccgccgggacgaggtgctgatcgccaccaagttcggcatggtgcgctcgtccgacggcgcctcccagggcttgtgcggcgagccgtcctacgtccgcgcggcctgcgaacggtccctgcgtcgtctcggcaccgaccgcatcgacctgtactaccagcactggacggacccggcggtgccgatcgaggagaccgtgggtgcggtggccgagctggtgcgcgagggcaaggtccgcaggctcggtctctccgagccctccgcggccacgctgcgccgggcggacgcggtgcacccggtgacggcggtgcagagcgagtggagcctgtggtcgcgcgggatcgaggacgaggtggtgcccgtctgccgggagctggggatcgggatcgtcgcttacgcccctctgggacggggttttctcaccggcaccatccgcaccaccgacgatctgggggacgaggacttccgccggggccagccccggttcagcgctccggccctcgcgcgcaaccgctcgttgctgcaccggctgcgcccggtcgcggacggtctggggctgaccctggcacagctcgcgctcgcctggctgcaccaccggggcgaggacgtcgtcccgatcccgggcaccgcgaacccggcccatctcgcggacaatctcgccgccgcctcgatccggctggacgaccggtccctcgcggaggtgacggccgcgatctcccacccggtgtccggggagcggtacaccccggcattgctcgccatgatcggcaac

SEQ ID NO: 5 - DNA, 984 bp, S. Clavuligerus
gtggaatgccgcatattcgagatcgacgaactgccgttgctggacggggaggtcctgcgggacgcccggatcggttacgccatgtacggcacgccgaacgccgacgggacgaacgtggtgctctgtccgtcgttcttcggccgggaccacaccgggtacgactggctgatcggtgcggggctgccgctggacacccggcggtactgcgtcgtcaccgccggactcttcggcaacggggtctccagctcgcccggcaaccacccgtcggggtcccgctttccgctgatcactccgcaggacaatgtcgcggcgcagcaccggctgctcaccgaggagctgggggtacgggaactggccctggtcacgggctggtcgatgggcgcggcccacgcctaccagtgggccgtgtcgcatccggggatggtgcgccggatcgccccgatctgcggggcgccggtgagcagcccgcacagcctggtcctgctgtccggtctggccgcggcgctcagcgccgacgccggggagcgggggcggaaggcggcgggccgggtgttcgccgggtgggggacctcgcgttccttctgggcccgccgtgcccaccgggagctgggtttcgccacccgcgaggagtacctcaccggcttctgggagcaggtcttcctctccgggcccggcgccgcggatctgctcaccatggtgcgcacctgggagaacacggatgtgggggcgacacccggggccggggggagcgtcgaggcggcgctggcctccgtcacggcgcgggccgtggtgctgccgggcgccctggacgtgtgtttcgccgtcgaggacgagaagcgggtggccgatctgctgccgtatgcctcgctggaggtgatcccgggagtgtgggggcatctcgcggggtccggggggtcggccgccgaccgggagttcatcgggggcgcgctgcggcggctgctggacagcccggtggacgggggc

SEQ ID NO: 6 - DNA, 1182 bp, S. Clavuligerus
gtgaagtccattctcttctatctgccaacggtcggcagtcatgcgcaggtccagcggggtatggcgggggtcaatccgcagaactaccagaacatgctccggcagctcacccggcaggcgcaggcggccgacgaactcggctactggggactgtccttcaccgagcaccacttccacaccgagggtttcgaggtctccaacaacccgatcatgctggggctctacctcggcatgcagacccggcacatccgggtcggccagatggccaacgtcctgccgctgcacaatccgctgcggctggccgaggatctggcgatgctcgaccacatgacccggggccgcgccttcgtcgggatcgcgcgcgggttccagaagcgctgggccgacatcatggggcaggtgtacggggtcggcggcaccctgtccgacgccggggagcgggaccggcgcaatcgtgccctcttcgaggagcactgggagatcatcaagaaggcgtggacgaccgagacgttcacccactccggggagcagtggacgatcccggtgccggacctggagttcccctacgaggcggtgcgccgctacggccggggcctcgacgagaacggcgtcatccgcgaggtgggcatcgcgcccaagccctaccagcgcccccacccgcccgtcttccagccgttcagcttcagtgaggacacgttccggttctgtgcccgggagggcgtggtgccgatcctgatgaacaccgacgaccagatcgtcgcccggctgatggacatctaccgggaggaggccgaggcggcgggccacggcaccctgcggcggggcgagcgggtcggggtgatgaaggacgtcctggtctcccgggactccggcgaggcccaccactgggcgtcccgcggcggcggcttcatcttcgagaactggttcggccccatgggcttcaccgaggcgctgcgcgcgaccggcgagacgggtccgatcggctcggactacaagaccctggtcgaccgggggctggagtgggtcggcaccccggacgacatcaaccgcatgatcgagaagctggtggagcggcacgatccggagtatctgctccagtgccagtactccgggctgatcccgcacgatgtccagctgcgcagcctggagctgtgggccaccgagatcgcccccaactggctc

SEQ ID NO: 7 - DNA, 660 bp, S. Clavuligerus
gtgcccggctccggactcgaagcactggaccgtgccaccctcatccaccccaccctctccggaaacaccgcggaacggatcgtgctgacctcggggtccggcagccgggtccgcgacaccgacggccgggagtacctggacgcgagcgccgtcctcggggtgacccaggtgggccacggccgggccgagctggcccgggtcgcggccgagcagatggcccggctggagtacttccacacctgggggacgatcagcaacgaccgggcggtggagctggcggcacggctggtggggctgagcccggagccgctgacccgcgtctacttcaccagcggcggggccgagggcaacgagatcgccctgcggatggcccggctctaccaccaccggcgcggggagtccgcccgtacctggatactctcccgccggtcggcctaccacggcgtcggatacggcagcggcggcgtcaccggcttccccgcctaccaccagggcttcggcccctccctcccggacgtcgacttcctgaccccgccgcagccctaccgccgggagctgttcgccggttccgacgtcaccgacttctgcctcgccgaactgcgcgagaccatcgaccggatcggcccggagcggatcgcggcgatgatcggcgagccgatc

SEQ ID NO: 8 - DNA, 25 bp, S. Clavuligerus
ctgacgctgcaggaggaagtcccgc

SEQ ID NO: 9 - DNA, 25 bp, S. Clavuligerus
cggggcgaggacgtcgtcccgatcc

SEQ ID NO: 10 - DNA, 25 bp, S. Clavuligerus
gagcccctggacgtcggcggtgtcc

SEQ ID NO: 11 - DNA, 25 bp, S. Clavuligerus
gacggtgcatgctcagcagggagcg

SEQ ID NO: 12 - DNA, 972 bp, S. Clavuligerus
atgacctcagtggactgcaccgcgtacggccccgagctgcgcgcgctcgccgcccggctgccccggaccccccgggccgacctgtacgccttcctggacgccgcgcacacagccgccgcctcgctccccggcgccctcgccaccgcgctggacaccttcaacgccgagggcagcgaggacggccatctgctgctgcgcggcctcccggtggaggccgacgccgacctccccaccaccccgagcagcaccccggcgcccgaggaccgctccctgctgaccatggaggccatgctcggactggtgggccgccggctcggtctgcacacggggtaccgggagctgcgctcgggcacggtctaccacgacgtgtacccgtcgcccggcgcgcaccacctgtcctcggagacctccgagacgctgctggagttccacacggagatggcctaccaccggctccagccgaactacgtcatgctggcctgctcccgggccgaccacgagcgcacggcggccacactcgtcgcctcggtccgcaaggcgctgcccctgctggacgagaggacccgggcccggctcctcgaccggaggatgccctgctgcgtggatgtggccttccgcggcggggtggacgacccgggcgccatcgcccaggtcaaaccgctctacggggacgcggacgatcccttcctcgggtacgaccgcgagctgctggcgccggaggaccccgcggacaaggaggccgtcgccgccctgtccaaggcgctcgacgaggtcacggaggcggtgtatctggagcccggcgatctgctgatcgtcgacaacttccgcaccacgcacgcgcggacgccgttctcgccccgctgggacgggaaggaccgctggctgcaccgcgtctacatccgcaccgaccgcaatggacagctctccggcggcgagcgcgcgggcgacgtcgtcgccttcacaccgcgcggc

SEQ ID NO: 13 - protein, 48 aa, S. Clavuligerus
MTRPPGLSAHTHGSVSGSLLRRVAGHYPTGVVLVTGPAEAPGQPPPAM

SEQ ID NO: 14 - protein, 151 aa, S. Clavuligerus
MSVASAGMTDEQRKAVITAYFKAFDNGGVGSDGTPAIDYFAEDAVFFFPKWGLARGKSEIARLFDDLGGTIRSITHHLWSVNWILTGTELLAAEGTTHGEHRDGPWRAGDPEWAAGRWCTVYEVRDFLVHRAFVYLDPDYAGKDTARYPWL

SEQ ID NO: 15 - protein, 344 aa, S. Clavuligerus
MSRSPPESPAGSVSAAVPRPPVRALRDLPVSAQGLGCLPTTDFYGRPDRARATATIRAAVDAGVTLLDTADVQGLGAGEELLGRAVAGRRDEVLIATKFGMVRSSDGASQGLCGEPSYVRAACERSLRRLGTDRIDLYYQHWTDPAVPIEETVGAVAELVREGKVRRLGLSEPSAATLRRADAVHPVTAVQSEWSLWSRGIEDEVVPVCRELGIGIVAYAPLGRGFLTGTIRTTDDLGDEDFRRGQPRFSAPALARNRSLLHRLRPVADGLGLTLAQLALAWLHHRGEDVVPIPGTANPAHLADNLAAASIRLDDRSLAEVTAAISHPVSGERYTPALLAMIGN

SEQ ID NO: 16 - protein, 328 aa, S. Clavuligerus
MECRIFEIDELPLLDGEVLRDARIGYAMYGTPNADGTNVVLCPSFFGRDHTGYDWLIGAGLPLDTRRYCVVTAGLFGNGVSSSPGNHPSGSRFPLITPQDNVAAQHRLLTEELGVRELALVTGWSMGAAHAYQWAVSHPGMVRRIAPICGAPVSSPHSLVLLSGLAAALSADAGERGRKAAGRVFAGWGTSRSFWARRAHRELGFATREEYLTGFWEQVFLSGPGAADLLTMVRTWENTDVGATPGAGGSVEAALASVTARAVVLPGALDVCFAVEDEKRVADLLPYASLEVIPGVWGHLAGSGGSAADREFIGGALRRLLDSPVDGG

SEQ ID NO: 17 - protein, 394 aa, S. Clavuligerus
MKSILFYLPTVGSHAQVQRGMAGVNPQNYQNMLRQLTRQAQAADELGYWGLSFTEHHFHTEGFEVSNNPIMLGLYLGMQTRHIRVGQMANVLPLHNPLRLAEDLAMLDHMTRGRAFVGIARGFQKRWADIMGQVYGVGGTLSDAGERDRRNRALFEEHWEIIKKAWTTETFTHSGEQWTIPVPDLEFPYEAVRRYGRGLDENGVIREVGIAPKPYQRPHPPVFQPFSFSEDTFRFCAREGVVPILMNTDDQIVARLMDIYREEAEAAGHGTLRRGERVGVMKDVLVSRDSGEAHHWASRGGGFIFENWFGPMGFTEALRATGETGPIGSDYKTLVDRGLEWVGTPDDINRMIEKLVERHDPEYLLQCQYSGLIPHDVQLRSLELWATEIAPNWL

SEQ ID NO: 18 - protein, 220 aa, S. Clavuligerus
MPGSGLEALDRATLIHPTLSGNTAERIVLTSGSGSRVRDTDGREYLDASAVLGVTQVGHGRAELARVAAEQMARLEYFHTWGTISNDRAVELAARLVGLSPEPLTRVYFTSGGAEGNEIALRMARLYHHRRGESARTWILSRRSAYHGVGYGSGGVTGFPAYHQGFGPSLPDVDFLTPPQPYRRELFAGSDVTDFCLAELRETIDRIGPERIAAMIGEPI

SEQ ID NO: 19 - protein, 324 aa, S. Clavuligerus
MTSVDCTAYGPELRALAARLPRTPRADLYAFLDAAHTAAASLPGALATALDTFNAEGSEDGHLLLRGLPVEADADLPTTPSSTPAPEDRSLLTMEAMLGLVGRRLGLHTGYRELRSGTVYHDVYPSPGAHHLSSETSETLLEFHTEMAYHRLQPNYVMLACSRADHERTAATLVASVRKALPLLDERTRARLLDRRMPCCVDVAFRGGVDDPGAIAQVKPLYGDADDPFLGYDRELLAPEDPADKEAVAALSKALDEVTEAVYLEPGDLLIVDNFRTTHARTPFSPRWDGKDRWLHRVYIRTDRNGQLSGGERAGDVVAFTPRG

(19) United States
(12) Patent Application Publication
(10) Pub. No.: US 2004/0081960 A1
(43) Pub. Date: Apr. 29, 2004

(54) Novel process

(75) Inventors: Cecilia Anders, Edmonton (CA); Barry Barton, Lancing (GB); John Patrick Griffin, Worthing (GB); Susan Jensen, Edmonton (CA); Roy Henry Mosher, Springfield, IL (US); Ashish Sudhakar Paradkar, Edmonton (CA)

Correspondence Address: GLAXOSMITHKLINE, Corporate Intellectual Property - UW2220, P.O. Box 1539, King of Prussia, PA, 19406-0939, US

(73) Assignees: SmithKline Beecham p.l.c.; The Governors of the University of Alberta

(21) Appl. No.: 10/138,222

(22) Filed: Feb. 24, 2003

Related U.S. Application Data
(63) Continuation of application No. 09/513,857, filed on Feb. 24, 2000, now abandoned.
Application No. 09/513,857 is a division of application No. 09/137,739, filed on Aug. 21, 1998, now abandoned.
Application No. 09/513,857 is a continuation of application No. 08/985,666, filed on Dec. 5, 1997, now abandoned.

(30) Foreign Application Priority Data
Feb. 4, 1997 (GB) 9702216.4

(51) Int. Cl. C12Q 1/68; C07H 21/04; C12P 17/18; C12N 1/21; C12N 15/74

(52) U.S. Cl. 435/6; 435/69.1; 435/119; 435/193; 435/252.3; 435/320.1; 536/23.2

(57) ABSTRACT

Novel bacterial genes, microorganisms and processes for improving the manufacture of 5R clavams, eg. clavulanic acid.

The present invention relates to novel bacterial genes and processes for improving the manufacture of clavams e.g. clavulanic acid. The present invention also provides novel organisms capable of producing increased amounts of clavulanic acid.

Microorganisms, in particular Streptomyces sp. produce a number of antibiotics including clavulanic acid and other clavams, cephalosporins, polyketides, cephamycins, tunicamycin, holomycin and penicillins. There is considerable interest in being able to manipulate the absolute and relative amounts of these antibiotics produced by the microorganism and accordingly there have been a large number of studies investigating the metabolic and genetic mechanisms of the biosynthetic pathways [Domain, A. L. (1990) “Biosynthesis and regulation of beta-lactam antibiotics.” In: 50 years of Penicillin applications, history and trends]. Many of the enzymes which carry out the various steps in the metabolic pathways and the genes which code for these enzymes are known.

Clavams can be arbitrarily divided into two groups dependent on their ring stereochemistry (5S and 5R clavams). The biochemical pathways for the biosynthesis of 5R and 5S clavams have not yet been fully elucidated but it has been suggested that they are derived from the same starter units (an as yet unidentified 3 carbon compound [Townsend, C. A. and Ho, M. F. (1985) J. Am. Chem. Soc. 107 (4), 1066-1068 and Elson, S. W and Oliver, R. S. (1978) J. Antibiotics XXXI No.6, 568] and arginine [Valentine, B. P et al (1993) J. Chem. Soc. Chem. Comm. 15, 1210-1211]) and share some common intermediates [Iwata-Reuyl, D. and C. A. Townsend (1992) J. Am. Chem. Soc. 114: 2762-63, Janc, J. W. et al (1993) Bioorg. Med. Chem. Lett 3:231316].

Examples of 5S clavams include clavam-2-carboxylate (C2C), 2 hydroxymethylclavam (2HMC), 2-(3-alanyl)clavam, valclavam and clavaminic acid (GB 1585661, Rohl, F. et al Arch. Microbiol. 147:315-320, U.S. Pat. No. 4,202,819) There are, however, few examples of 5R clavams and by far the most well known is the beta lactamase inhibitor clavulanic acid which is produced by the fermentation of Streptomyces clavuligerus. Clavulanic acid is an active component of the antibiotic AUGMENTIN (Trade Mark SmithKline Beecham). Because of this commercial interest, investigations into the understanding of clavam biosynthesis have concentrated on the biosynthesis of the 5R clavam, clavulanic acid, by S. clavuligerus. A number of enzymes and their genes associated with the biosynthesis of clavulanic acid have been identified and published. Examples of such publications include Hodgson, J. E. et al Gene 166. 49-55 1995, Aidoo, K. A. et al Gene 147, 41-46 1994, Paradkar, A. S. et al J. Bact. 177.5.1307-14 1995. In contrast nothing is known about the biosynthesis and genetics of 5S clavams other than clavaminic acid which is a clavulanic acid precursor produced by the action of clavaminic acid synthase in the clavulanic acid biosynthetic pathway in S. clavuligerus.

Gene cloning experiments have identified that S. clavuligerus contains two clavaminic acid synthase isoenzymes, cs1 and cs2 (Marsh, E. N. et al Biochemistry 31, 12648-657 1992) both of which can contribute to clavulanic acid production under certain nutritional conditions (Paradkar, A. S. et al J. Bact. 177.5.1307-14 1995). Clavaminic acid synthase activity has also been detected in other clavulanic acid producing micro-organsims, ie. S. jumonjinensis (Vidal, C. M ES 550549, 1987) and S. katsurahamanus (Kitano, K et al JP 53-104796, 1978) as well as S. antibioticos, a producer of the 5S clavam valclavam (Baldwin, J. E. et al Tetrahedron Letts. 35,17,2783-86, 1994). The latter paper also reported S. antibioticos to have proclavaminic acid amidino hydrolase activity, another enzyme known to be involved in clavulanic acid biosynthesis. All other genes identified in S. clavuligerus as involved in clavam biosynthesis have been reported to be required for clavulanic acid biosynthesis (Hodgson, J. E. et al Gene 166. 49-55 1995, Aidoo, K. A. et al Gene 147, 41-46 1994) and as yet none have been reported which are specific for the biosynthesis of 5S clavams.

We have now identified certain genes which are specific for the biosynthesis of 5S clavams as exemplified by C2C and 2HMC in S. clavuligerus. Accordingly the present invention provides DNA comprising one or more genes which are specific for 5S clavam biosynthesis in S. clavuligerus and which are not essential for 5R clavam (e.g. clavulanic acid) biosynthesis.

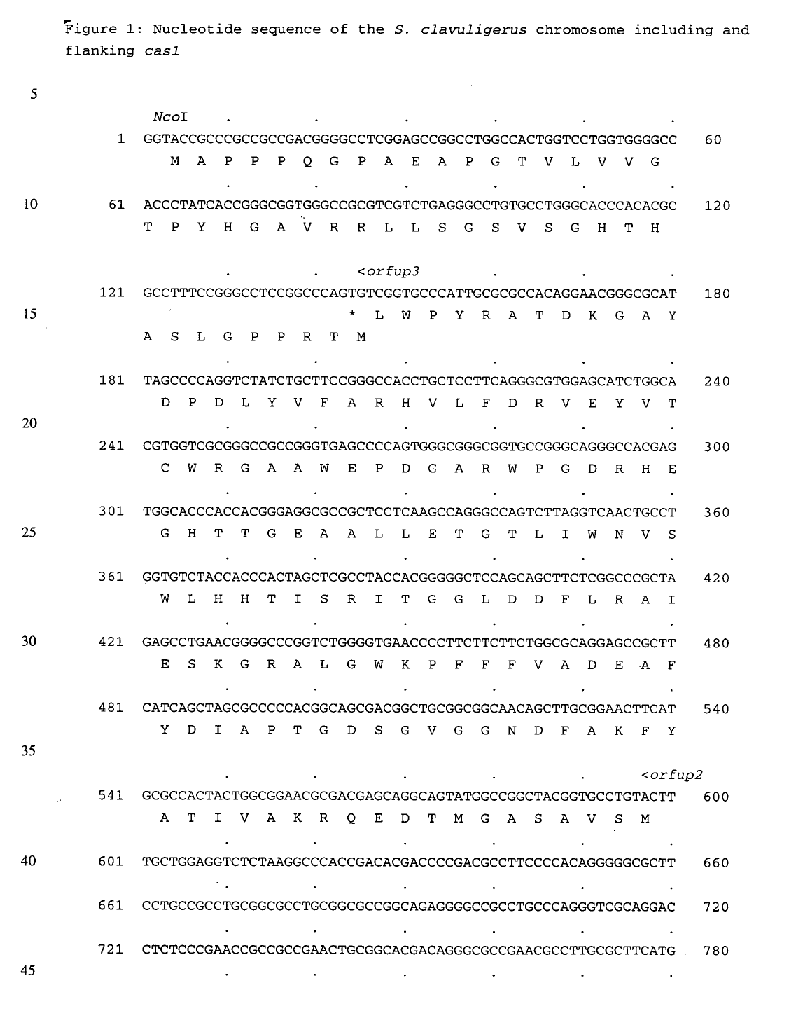

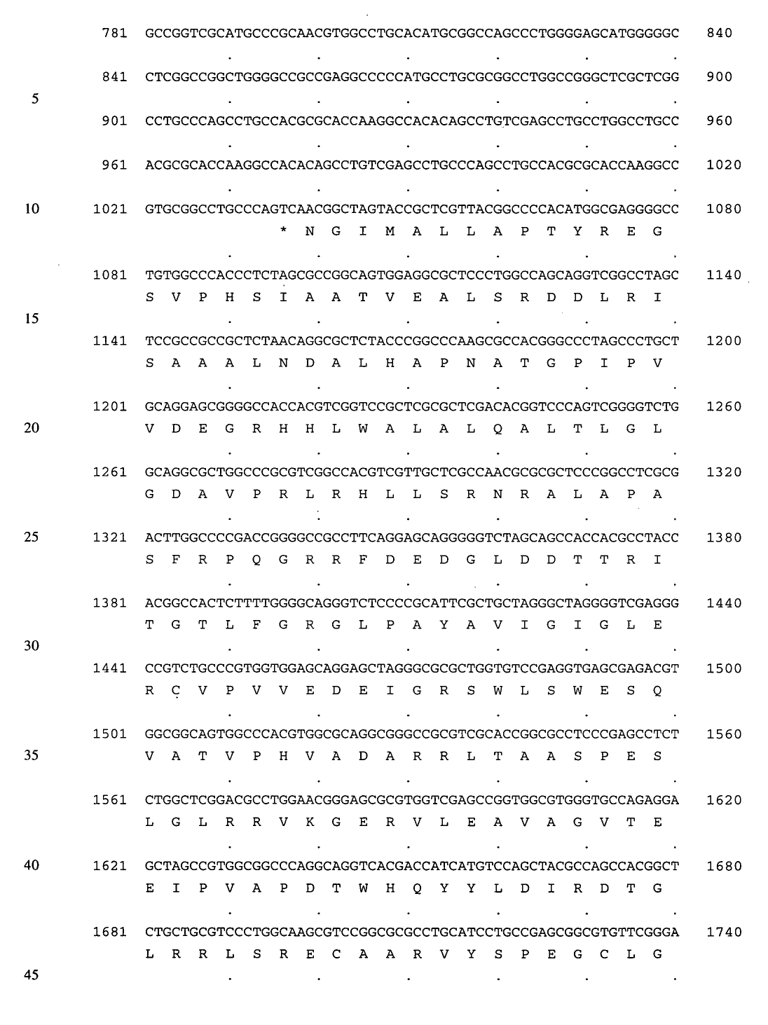

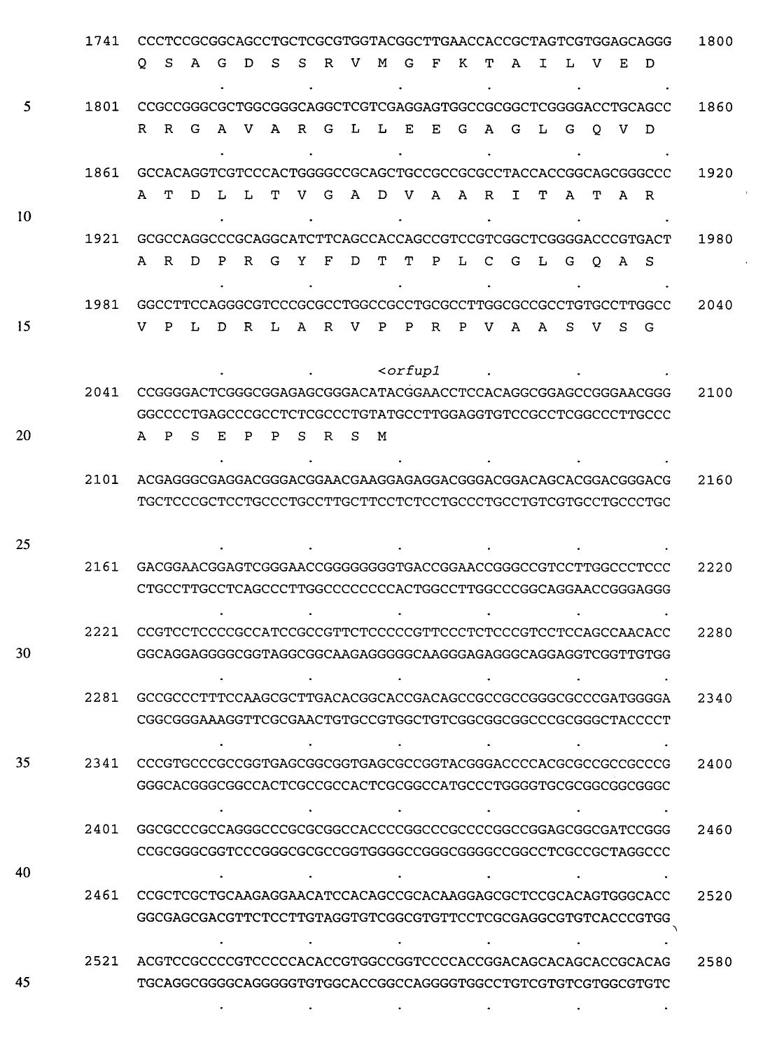

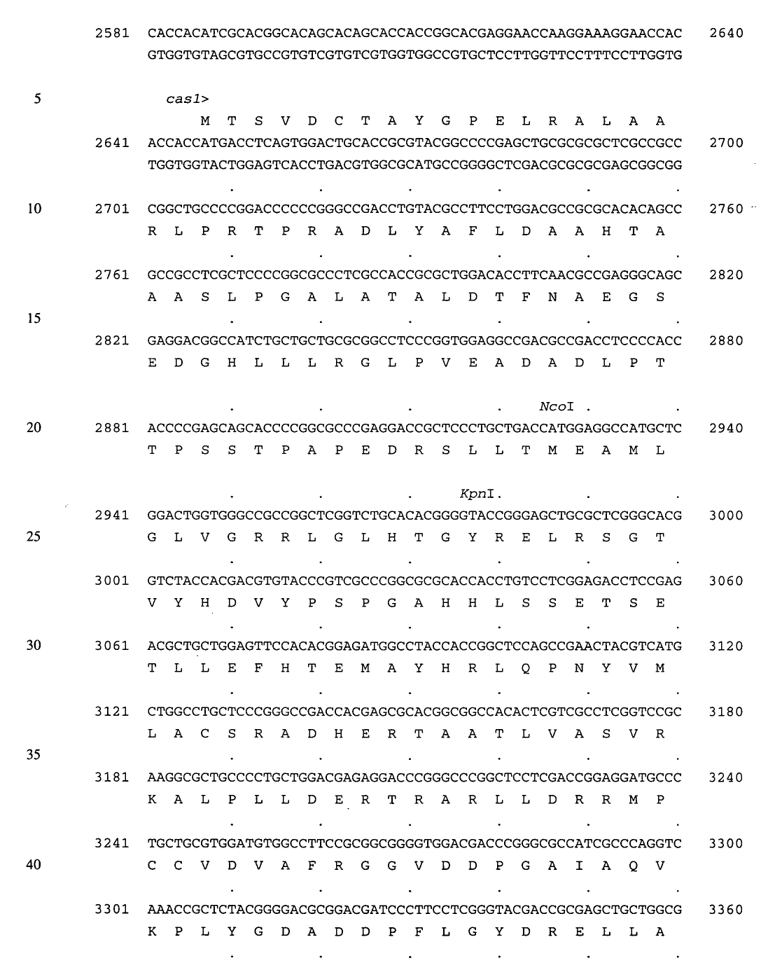

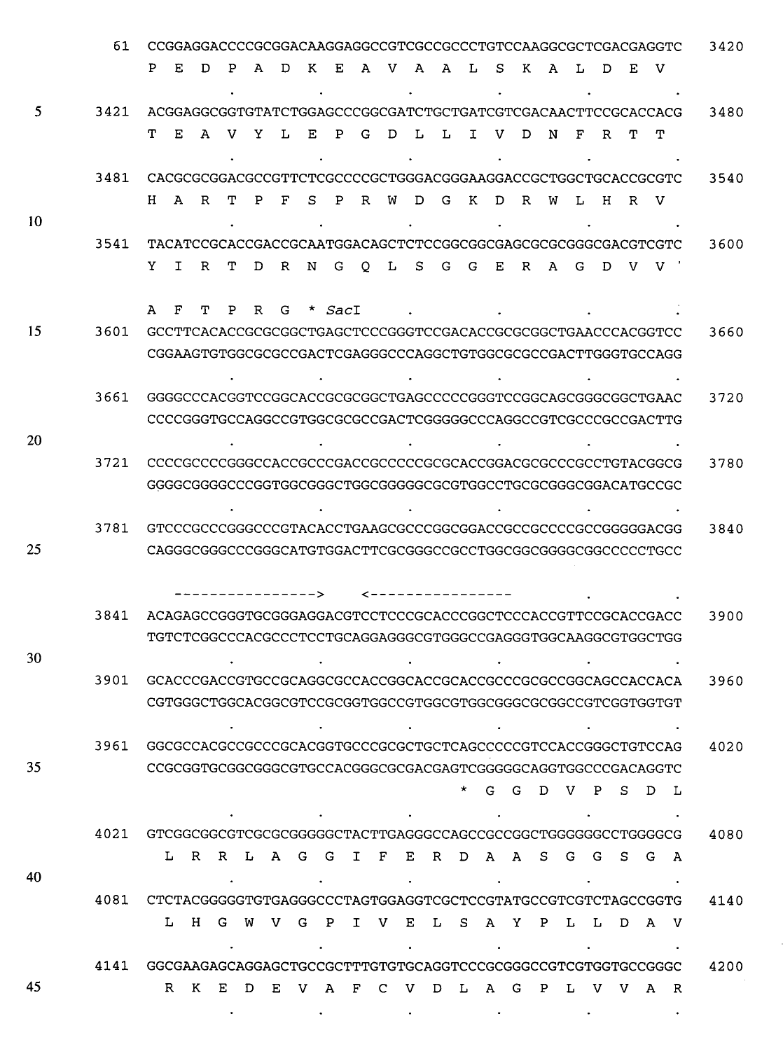

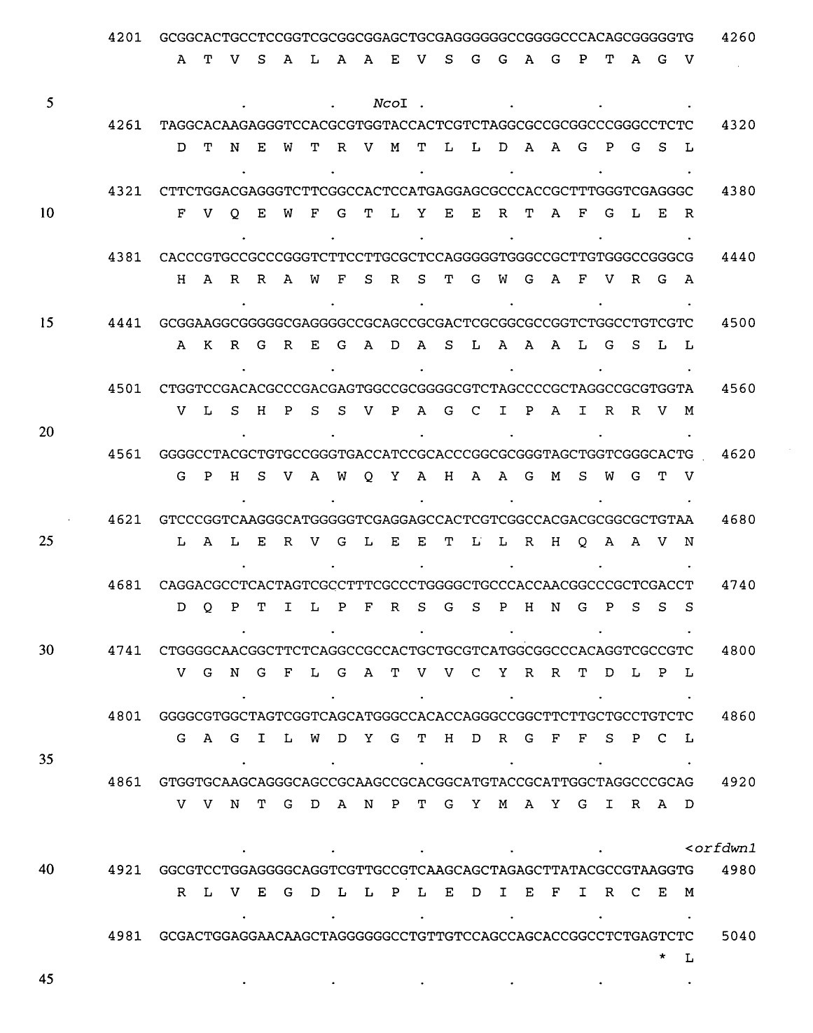

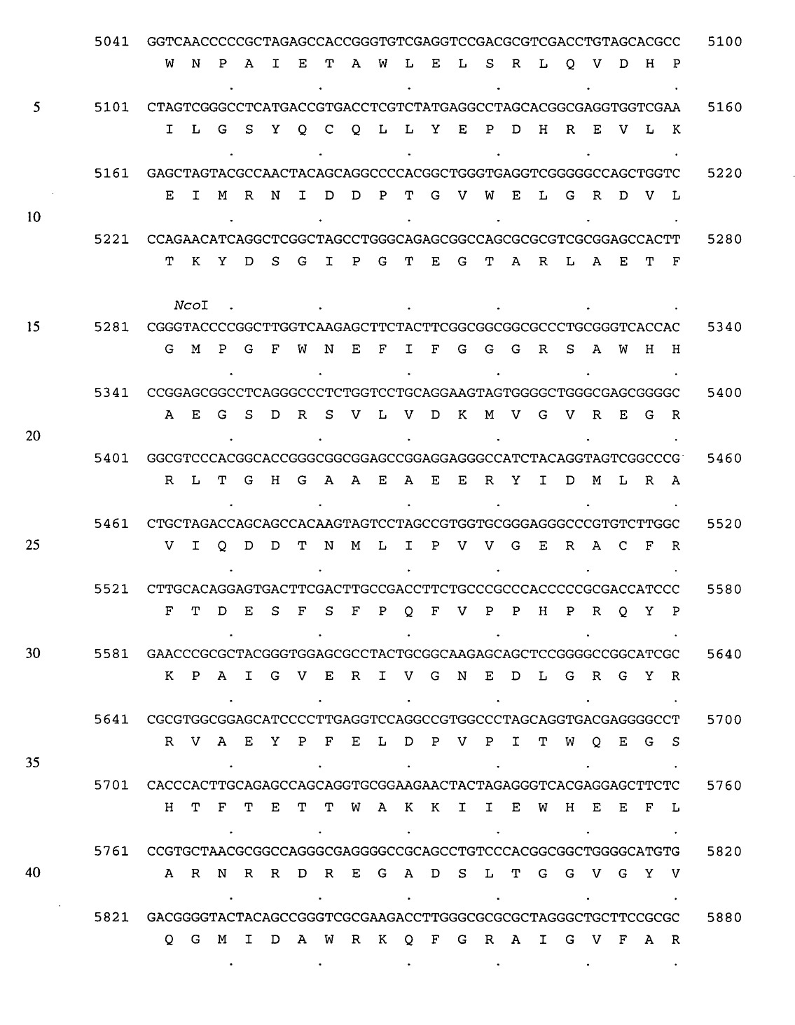

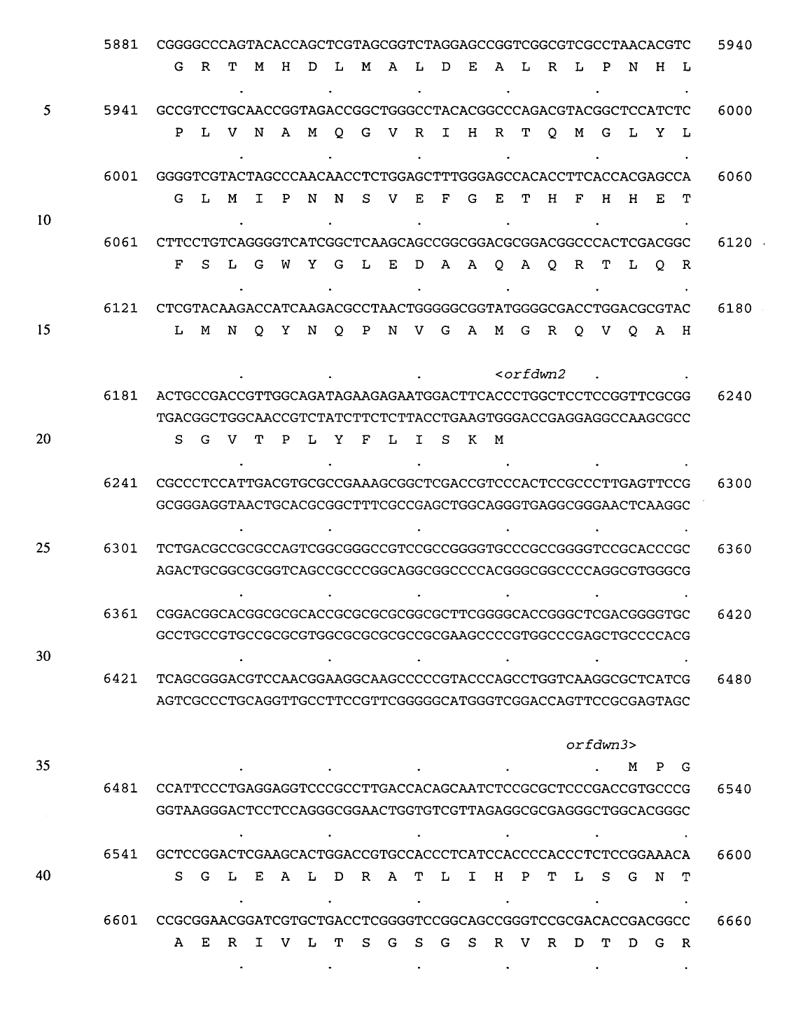

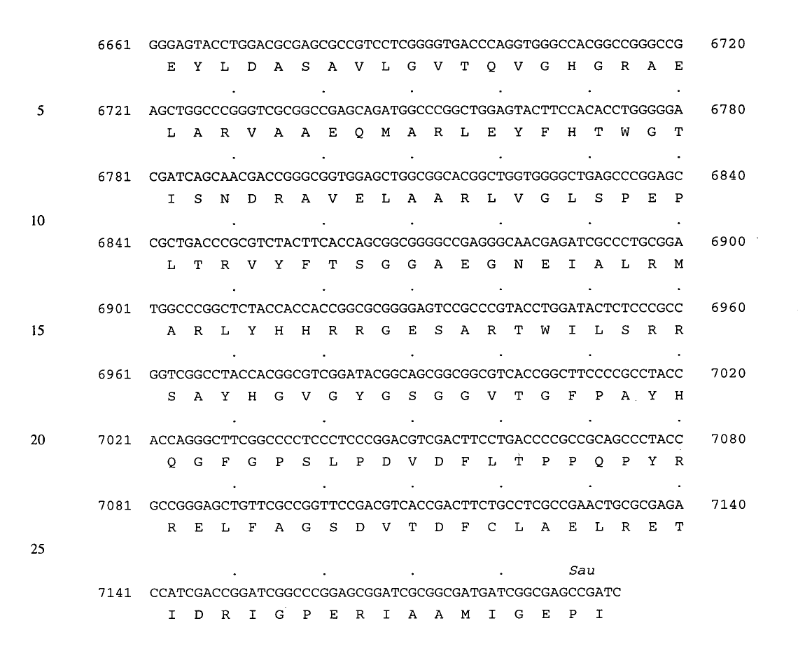

By “gene” as used herein we also include any regulatory region required for gene function or expression. In a preferred aspect the DNA is as identified as FIG. 1. Preferably the DNA comprises the nucleotide sequences indicated in FIG. 1 designated as orfup3, orfup2, orfup1, orfdwn1, orfdwn2 and orfdwn3. The present invention also provides proteins coded by said DNA. The present invention also provides vectors comprising the DNA of the invention and hosts containing such vectors. Surprisingly we have found that when at least one of the genes according to the invention is defective the amount of clavulanic acid produced by the organism is increased.

Accordingly the present invention also provides processes for increasing the amount of clavulanic acid produced by a suitable microorganism. In one aspect of the invention the genes identified can be manipulated to produce an organism capable of producing increased amounts of clavam, suitably clavulanic acid. The findings of the present work also allow an improved process for the identification of organisms with higher clavulanic acid production comprising a preliminary screening for organisms with low or no 5S clavam production (for example by hplc and/or clavam bioassay as described in the examples herein).

Suitably the 5S clavam genes of the present invention can be obtained by conventional cloning methods (such as PCR) based on the sequences provided herein. The function of the gene can be interfered with or eliminated/deleted by genetic techniques such as gene disruption [Aidoo, K. A. et al., (1994), Gene, 147, 41-46]., random mutagenesis, site directed mutagenesis and antisense RNA.

In a further aspect of the invention there are provided plasmids containing one or more defective genes, preferably the plasmids pCEC060, pCEC061, pCEC056 and pCEC057, described below. Suitably the plasmids are used to transform an organism such as S. clavuligerus e.g. strain ATCC 27064 (which corresponds to S. clavuligerus NRRL 3585).

Suitable transformation methods can be found in relevant sources including Sambrook, J., E. F. Fritsch, and T. Maniatis (1989) Molecular cloning: a laboratory manual, 2nd Ed. Cold Spring Harbor Laboratory, Cold Spring Harbor, N.Y.; Hopwood, D. A. et al. (1985) Genetic Manipulation of Streptomyces. A Cloning Manual, and Paradkar, A. S. and Jensen, S. E. (1995) J. Bacteriol. 177 (5): 1307-1314.

Strains of the species S. clavuligerus are used industrially to produce clavulanic acid (potassium clavulanate). Within the British and United States Pharmacopoeias for potassium clavulanate (British Pharmacopoeia 1993, Addendum 1994 p1362-3 and U.S. Pharmacopeia Official Monographs 1995 USP 23 NF18 p384-5) the amounts of the toxic 5S clavam, clavam-2-carboxylate, are specifically controlled.

Therefore in a further aspect of the invention there is provided an organism capable of producing high amounts of clavulanic acid but has been made unable to make C2C. Suitably the clavulanic acid producing organism contains one or more defective clavam genes, and is preferably the S. clavuligerus strains 56-1A, 56-3A, 572B, 57-1C, 60-1A, 60-2A, 60-3A, 61-1A, 61-2A, 61-3A, and 61-4A, described below. Such organisms are suitable for the production of clavulanic acid without the production of the 5S clavam, clavam-2-carboxylate.

The S. clavuligerus strains (56-1A, 56-3A, 57-2B, 57-1C, 60-1A, 60-2A, 603A, 61-1A, 61-2A, 61-3A, and 61-4A were deposited with NCIMB on Dec. 11, 1996 in accordance with the Budapest Treaty on the International recognition of the Deposition of Microorganisms for the Purposes of Patent Procedure (1977) and Implementing Regulations (1981). The NCIMB designation for each is shown in the table below.

| STRAIN/PLASMID | NCIMB ACCESSION No. |
|---|---|
| 56-1A | NCIMB 40838 |
| 56-3A | NCIMB 40839 |
| 57-2B | NCIMB 40840 |
| 57-1C | NCIMB 40841 |
| 60-1A | NCIMB 40842 |
| 60-2A | NCIMB 40843 |
| 60-3A | NCIMB 40844 |
| 61-1A | NCIMB 40845 |
| 61-2A | NCIMB 40846 |
| 61-3A | NCIMB 40847 |
| 61-4A | NCIMB 40848 |

### EXAMPLES

In the examples all methods are as in Sambrook, J., Fritsch, E. F. and Maniatis, T. (1989) Molecular Cloning A Laboratory Manual (2nd Edition), or Hopwood, D. A. et al. (1985) Genetic Manipulation of Streptomyces. A Cloning Manual, and Paradkar, A S. and Jensen, S. E. (1995) J. Bacteriol. 177 (5): 1307-1314 unless otherwise stated.

I. DNA Sequencing of the Streptomyces clavuligerus Chromosome Upstream and Downstream of the Clavaminate Synthase Gene cas1.

A. Isolation of cas1.

To isolate chromosomal DNA fragments from Streptomyces clavuligerus NRRL 3585 encoding the gene for clavaminate synthase isozyme 1 (cas1) an oligonucleotide probe RMO1 was synthesised based on nucleotides 9-44 of the previously sequenced cas1 gene (Marsh, E. N., M. D. T. Chang, and C. A. Townsend (1992) Biochemistry 31: 12648-12657). Oligonucleotides were constructed using standard methods on an Applied Biosystems 391 DNA Synthesiser. The sequence of RMO1, a 36-mer, was synthesised in the antiparallel sense to that published by Marsh et al (1992, ibid) RMO1 was radiolabelled with ³²p using standard techniques for end-labelling DNA oligonucleotides (Sambrook et al., 1989 ibid), and was used to screen a cosmid bank of Streptomyces clavuligerus genomic DNA by Southern hybridization as described by Stahl and Amann (In: Nucleic acid techniques in bacterial systematics. Ed. E. Stackebrandt and M. Goodfellow. Toronto: John Wiley and Sons, p. 205-248, 1991). The genomic bank of S. clavuligerus DNA, prepared in cosmid pLAFR3, was as described by Doran, J. L et al (1990) J. Bacteriol. 172 (9), 4909-4918.

Colony blots of the S. clavuligerus cosmid bank were incubated overnight with radiolabelled RMO1 at 60° C. in a solution consisting of 5×SSC, 5× Denhardt's solution, and 0.5% SDS (1×SDS: 0.15 M NaCl+0.015 M Na₃citrate; 1× Denhardt's solution: 0.02% BSA, 0.02% Ficoll, and 0.02% PVP). The blots were then washed at 68° C. for 30 minutes in a solution of 0.5×SSC+0.1% SDS. One cosmid clone, 10D7, was isolated that hybridised strongly to RMO1 and gave hybridization signals upon digestion with restriction endonucleases SacI and EcoRI that were consistent with hybridization signals detected in similar experiments with digests of S. clavuligerus genomic DNA.

B. DNA Sequencing of the S. clavuligerus Chromosome Flanking cas1.

A partial restriction map of cosmid 10D7 was generated using restriction endonucleases SacI, NcoI, and KpnI. Southern hybridization experiments between RMO1 and various digests of 10D7 DNA indicated that cas1 was most likely located at one end of a 7-kb SacI-SacI DNA subfragment. This fragment consisted of the cas1 open reading frame and approximately 6 kb of upstream DNA. The 7-kb fragment was then subcloned from a SacI digest of 10D7 in the phagemid vector pBluescriptII SK+ (2.96 kb; Stratagene), thus generating the recombinant plasmid pCEC007.

To facilitate the process of sequencing the chromosome upstream of cas1, a 3 kb NcoI-NcoI subfragment of the 7-kb SacI-SacI fragment was subcloned in pUC120 (3.2 kb; Vieirra and Messing, Methods Enzymol. 153, 3-11, 1987)) in both orientations, generating the recombinant plasmids pCEC026 and pCEC027. The 3-kb subfragment consisted of the amino-terminal-encoding portion of cas1 and approximately 2.6 kb of upstream DNA.

Nested, overlapping deletions were created in both pCEC026 and pCEC027 using exonuclease III and S1 nuclease digestion (Sambrook et al., 1989 ibid) and the DNA sequence of the 3-kb NcoI-NcoI fragment was determined on both strands by the dideoxy chain termination method (Sanger, F., S. Nicklen, and A. R. Coulson (1977) Proc. Natl. Acad. Sci. U.S.A. 74: 5463-5467.) using a Taq dye-deoxy^{a} terminator kit and an Applied Biosystems 373A Sequencer.

To determine the DNA sequence of the chromosome immediately downstream of cas1 a 4.3-kb KpnI-EcoRI DNA fragment was subcloned from cosmid clone 10D7 in pBluescriptII SK+, generating pCEC018. From pCEC018 a 3.7-kb SacI-SacI subfragment was cloned in pSL1180 (3.422 kb, Pharmacia); one of the SacI termini of this fragment partially overlapped the TGA stop codon of cas1, the other was vector encoded. Both orientations of the 3.7-kb fragment were obtained during subcloning and the resulting recombinant plasmids were designated pCEC023 and pCEC024. Nested, overlapping deletions were created in both plasmids and the DNA sequence of the 3.7-kb fragment was determined on both strands. The nucleotide sequence of the S. clavuligerus chromosome generated in these experiments, including and flanking cas1 sequence is shown in FIG. 1.

II. Functional Analysis of the Open Reading Frames Flanking cas1.

Computer analysis of the DNA sequence upstream of cas1 predicted the presence of two complete orfs and one incomplete orf. All three orfs were located on the opposite DNA strand to cas1 and were thus oriented in the opposite direction. The first open reading frame, orfup1, was located 579 bp upstream of cas1 and encoded a polypeptide of 344 amino acids (aa). The second open reading frame, orfup2, was located at 437 bp beyond the 3′-end of orfup1 and encoded a 151 aa polypeptide. Beyond orfup2 is orfup3. The start codon of orfup3 overlaps the translational stop codon of orfup2, suggesting that the two orfs are translationally coupled. No translational stop codon for orfup3 was located on the 3-kb NcoI-NcoI fragment.

A similar analysis of the DNA sequence downstream of cas1 predicted the presence of two complete orfs and one incomplete orf. Two of the orfs e located on the opposite DNA strand to cas1 and were thus oriented towards cas1. The third orf was located on the same strand as cas1 and was thus oriented away from it. The first downstream open reading frame, orfdwn1, was located 373 bp downstream of cas1 and encoded a 328 aa polypeptide. The second open reading frame, orfdwn2, was located 55 bp upstream of orfdwn1 and encoded a 394 aa polypeptide. At 315 bp upstream of orfdwn2 and on the opposite strand was orfdwn3. Because no stop codon was observed for orfdwn3 on the 3.7-kb fragment, it encoded an incomplete polypeptide of 219 aa.

Gene Disruption of the orfup and orfdwn Open Reading Frames

To assess the possible roles of the open reading frames flanking cas1 in the biosynthesis of clavulanic acid and the other clavams produced by S. clavuligerus, insertional inactivation or deletion mutants were created by gene replacement. The method used for gene disruption and replacement was essentially as described by Paradkar and Jensen (1995 ibid).

A. orfup1

A 1.5-kb NcoI-NcoI fragment carrying the apramycin resistance gene (apr^{r}), constructed as described in Paradkar and Jensen (1995 ibid), was treated with Klenow fragment to generate blunted termini (Sambrook et al., 1989 ibid) and was ligated to pCEC026 that had been digested with BsaBI and likewise treated with Klenow fragment. pCEC026 possesses a BsaBI site located within orfup1 at 636 bp from the translational start codon. The ligation mixture was used to transform competent cells of E. coli GM 2163 (available from New England Biolabs, USA., Marinus, M. G. et al M G G (1983) vol 122, p288-9) to apramycin resistance. From the resulting transformants two clones containing plasmids pCEC054 and pCEC055 were isolated; by restriction analysis pCEC054 was found to possess the apr^{r}-fragment inserted in the same orientation as orfup1, while pCEC055 possessed it in the opposite orientation.

To introduce pCEC054 into S. clavuligerus, plasmid DNA was digested with BamHI and HindIII and ligated to the high-copy number Streptomyces vector pU486 (6.2 kb; Ward et al., (1986) Mol. Gen. Genet. 203: 468-478). The ligation mixture was then used to transform E. coli GM2163 competent cells to apramycin resistance. From the resulting transformants one clone, possessing the shuttle plasmid pCEC061, was isolated. This plasmid was then used to transform S. clavuligerus NRRL 3585. The resulting transformants were put through two successive rounds of sporulation on non-selective media and then replica plated to antibiotic containing media to identify apramycin-resistant and thiostrepton-sensitive transformants. From this process four putative mutants (61-1A, -2A, -3A and -4A) were chosen for further analysis.

To confirm that these putative mutants were disrupted in orfup1 genomic DNA was prepared from isolates 61-1A and 61-2A, digested with SacI and subjected to Southern blot analysis. The results of the Southern blot were consistent with a double cross-over having occurred and demonstrated that these mutants are true disruption replacement mutants in orfup1.

The mutants 61-1A, -2A, -3A and -4A were grown in Soya-Flour medium and their culture supernatants were assayed by HPLC for clavulanic acid and clavam production. The composition of the Soya-Flour medium and the method for assaying clavams by HPLC were as previously reported (Paradkar and Jensen, 1995 ibid) except that the running buffer for the HPLC assay consisted of 0.1 M NaH₂PO₄+6% methanol, pH 3.68 (adjusted with glacial acetic acid). The HPLC analysis indicated that none of the mutants produced detectable levels of clavam-2-carboxylate or 2-hydroxymethylclavam. Furthermore, when culture supernatants were bioassayed against Bacillus sp. ATCC 27860, using the method of Pruess and Kellett (1983, J. Antibiot. 36: 208-212)., none of the mutants produced detectable levels of alanylclavam. In contrast, HPLC assays of the culture supernatants showed that the mutants appeared to produce superior levels of clavulanic acid when compared to the wild-type (Table 1).

TABLE 1
Clavulanic acid titre (CA) of orfup1 mutants in shake flask tests

| | 70 | 70 | 93 | 93 |
|---|---|---|---|---|
| | HOURS | HOURS | HOURS | HOURS |
| | CA | CA | CA | CA |
| STRAIN | ug/ml | ug/mg DNA | ug/ml | ug/mg DNA |
| NRRL 3585 #1 | 87 | 915 | 166 | 1963 |
| NRRL 3585 #2 | 66 | 790 | 159 | 1842 |
| 61-1A | 272 | 2894 | 439 | 6113 |
| 61-2A | 199 | 2148 | 225 | 2928 |
| 61-3A | 54 | 692 | 221 | 2585 |
| 61-4A | 0 | 0 | 226 | 2422 |

B. orfdwn1 and orfdwn2

A deletion/replacement mutant in orfdwn1 and orfdwn2 was created by first digesting pCEC018 (7.3 kb) with NcoI and liberating a 1-kb subfragment containing most of orfdwn1 and a portion of orfdwn2. The digest was fractionated by agarosegel electrophoresis and the 6.3-kb fragment was excised and eluted from the gel. This fragment was then ligated to an NcoI-NcoI DNA fragment carrying apr^{r} and used to transform E. coli XL1-Blue to apramycin resistance. One clone was obtained from this experiment but restriction analysis of the resulting recombinant plasmid revealed that two copies of the apramycin resistance fragment had been ligated into the deletion plasmid. To eliminate the extra copy of the apr^{r}-fragment, the plasmid was digested with NcoI and self-ligated. The ligation mixture was used to transform E. coli GM2163 to apramycin resistance. From the transformants, two clones were isolated that contained plasmids pCEC052 and pCEC053 both of which possessed only one copy of the apr^{r}-fragment; pCEC052 possessed the apr^{r}-fragment inversely oriented with respect to orfdwn1 and 2, while pCEC053 possessed the apr^{r}-fragment inserted in the same orientation as orfdwn1 and 2.

A shuttle plasmid of pCEC052 was constructed by ligating BamHI-digested pCEC052 with similarly digested pU486 and transforming E. coli GM2163 to apramycin resistance. From this experiment one clone was isolated that contained the shuttle plasmid pCEC060. This plasmid was used to transform wild-type S. clavuligerus 3585 to apramycin and thiostrepton resistance. The resulting transformants were put through two rounds of sporulation under non-selective conditions and then replica plated to antibiotic containing media to identify apramycin resistant, thiostrepton sensitive colonies. Three putative mutants (60-1A, -2A and -3A) were chosen for further analysis.

To establish the identity of these putative mutants genomic DNA was isolated from strains 60-1A and 60-2A and digested with either SacI or BstEII and subjected to southern blot analysis. The hybridisation bands generated from this experiment were consistent with both strains having undergone a double cross-over event demonstrating that these mutants are true disruption replacement mutants in orfdwn1/2.

When these were cultured in Soya-Flour medium and their culture supernatants assayed by HPLC, none of the mutants produced detectable levels of clavam-2-carboxylate or 2-hydroxymethylclavam. A bioassay of the culture supernatants showed that the mutants also failed to produce detectable levels of alanylclavam. As with the orfup1 mutants the orfdwn12 mutants are capable of producing superior to wild-type levels of clavulanic acid (Table 2).

TABLE 2
Clavulanic acid titre (CA) of orfdwn1/2 mutants in shake flask tests

| | 70 | 70 | 93 | 93 |
|---|---|---|---|---|
| | HOURS | HOURS | HOURS | HOURS |
| | CA | CA | CA | CA |
| STRAIN | ug/ml | ug/mg DNA | ug/ml | ug/mg DNA |
| NRRL 3585 #1 | 87 | 915 | 166 | 1963 |
| NRRL 3585 #2 | 66 | 790 | 159 | 1842 |
| 60-1A | 164 | 1872 | 260 | 2911 |
| 60-2A | 187 | 2013 | 108 | 1320 |
| 60-3A | 79 | 994 | 214 | 2161 |

orfdwn3

To disrupt orfdwn3 pCEC023 (consisting of a 3.7-kb fragment of cas1 downstream DNA subcloned into pSL1180) was digested with NcoI and then self ligated. After transforming E. coli with the ligation mixture a clone was isolated that possessed the plasmid pCEC031. This plasmid retained only the 1.9 kb NcoI-EcoRI fragment encoding a portion of orfdwn2 and the incomplete orfdwn3. An examination of the DNA sequence revealed that pCEC031 possessed a unique BstEII site at 158 bp from the translational start site of orfdwn3. Therefore, pCEC031 was digested with BstEII, treated with Klenow fragment to create blunt ends and then ligated to a blunted apramycin resistance cassette. The ligation mixture was used to transform E. coli GM2163 to apramycin resistance and ampicillin resistance. Two transformants were selected that contained respectively pCEC050 and pCEC051. restriction analysis revealed that the apramycin resistance cassette was orientated in the same orientation as orfdwn3 in pCEC050 and in the opposite orientation in pCEC051. Both of these plasmids were then digested with HindIII and ligated to similarly digested pIJ486. The ligation mixtures were then used separately to transform E. coli GM2163 to apramycin and ampicillin resistance. The shuttle plasmids pCEC056 (pCEC050+pIJ486) and pCEC057 (pCEC051+pU486) were isolated from the resultant transformants. Both plasmids were then used to transform S. clavuligerus NRRL 3585.

One transformant was selected from each transformant experiment and put through two successive rounds of sporulation on non-selective media and then replica plated to antibiotic containing media to identify apramycin-resistant and thiostrepton-sensitive transformants. From this process two putative mutants were isolated from the progeny of each primary transformant. (56-1A and 56-3A for pCEC056, and 571C and 57-2B for pCEC057).

To establish the identity of these putative mutants genomic DNA was isolated from these strains and digested with either SacI or Acc65I and subjected to Southern blot analysis. The hybridisation bands generated from this experiment were consistent with both strains having undergone a double cross-over event demonstrating that these mutants are true disruption replacement mutants in orfdwn3.

When these strains were cultured in Soya-Flour medium and their culture supernatants assayed by HPLC, the mutants produced greatly reduced levels of clavam-2-carboxylate or 2-hydroxymethylclavam. A bioassay of the culture supernatants showed that the mutants also failed to produce detectable levels of alanylclavam. As with the orfup1 and orfdwn1/2 mutants, the orfdwn3 mutant swere capable of producing superior to wild-type levels of clavulanic acid (Table 3).

TABLE 3
Clavulanic acid titre (CA) of orfdwn3 mutants in shake flask tests

| | 71 | 71 | 93 | 93 |
|---|---|---|---|---|
| | HOURS | HOURS | HOURS | HOURS |
| | CA | CA | CA | CA |
| STRAIN | ug/ml | ug/mg DNA | ug/ml | ug/mg DNA |
| NRRL 3585 | 180 | 1580 | 193 | 1790 |
| #1A | | | | |
| NRRL 3585 | 179 | 1640 | 266 | 2310 |
| #1B | | | | |
| 56-1A | 34 | 110 | 235 | 2160 |
| 56-3A | 225 | 2140 | 274 | 2740 |
| 57-1C | 253 | 2910 | 277 | 2920 |
| 57-2B | 242 | 2240 | 193 | 1860 |

1. DNA comprising one or more genes specific for 5S clavam biosynthesis in S. clavuligerus and which is not essential for 5R clavam biosynthesis.

2. DNA according to claim 1 as identified in FIG. 1.

3. DNA according to claim 1 having the sequence or substantially the sequence shown in FIG. 1 as orfup3, orfup2, orfup1, orfdwn1, orfdwn2 or orfdwn3.

4. DNA which hybridises under conditions of high stringency with the DNA of any one of claims 1 to 3

5. A vector comprising the DNA of claims 1 in which one or more of the genes have been disrupted.

6. A host comprising the vector of claim 5.

7. A host according to claim 6 which is capable of producing raised levels of clavulanic acid.

8. A host according to claim 7 which is capable of producing low or no levels of 5S clavam.

9. S. clavuligeris comprising DNA corresponding to open reading frames flanking cas1 which DNA contains a disruption.

10. S. clavuligeris of claim 9 wherein the disruption-containing DNA corresponding to open reading frames flanking cas1 is not essential for the production of 5R clavam.

11. S. clavuligeris of claim 10 wherein the open reading frames are selected from the group consisting of orfup3, orfup2, orfup 1, orfdwn1, orfdwm2 and orfdwm3.

12. A process for improving 5R clavam production in a suitable microorganism comprising manipulation of DNA of claim 1 and its inclusion in the said microorganism.

13. A process for improving 5R clavam production by S. clavuligeris comprising disrupting DNA regions flanking cas1.

14. A process for improving 5R clavam production by S. clauviligeris comprising disrupting DNA flanking cas1, said DNA being selected from the group consisting of orfup3, orfup2, orfup 1, orfdwn1, orfdwm2 and orfdwm3

15. A process for the identification of organisms suitable for high clavulanic acid production comprising a preliminary screening for organisms with low or no 5S clavam production.

16. A process according to claim 12 wherein the organism is S. clavuligerus.

17. A process according to claim 16 wherein the 5R clavam is clavulanic acid.

18. A process according to claim 17 wherein one or more genes for the production of 5S clavams are defective.

19. A microorganism obtainable by the process of claim 15.

20. A microorganism obtainable by the process of claim 15 which is capable of producing clavulanic acid but which does not produce clavam-2-carboxylate and/or 2hydroxymethylclavam.

21. A microorganism obtained by the process of claim 15 which is NCIMB 40838, 40839, 40840, 40841, 40842, 40843, 40844, 40845, 40846, 40847 or 40848.

22. Clavulanic acid obtained by the fermentation of the microrganism of claim 19.

23. Clavulanic acid according to claim 22 free of clavam-2-carboxylate.
